(19) United States
(12) Patent Application Publication
(10) Pub. No.: US 2005/0215959 A1
(43) Pub. Date: Sep. 29, 2005

(54) Delivery devices and methods of delivering liquids and nutrition to patients

(75) Inventor: Peter F. Whitington, Chicago, IL (US)

Correspondence Address: Brinks Hofer Gilson & Lione, P.O. Box 10395, Chicago, IL, 60610, US

(73) Assignee: Children's Memorial Hospital.

(21) Appl. No.: 10/809,741

(22) Filed: Mar. 24, 2004

(51) Int. Cl. A61M 31/00

(52) U.S. Cl. 604/278; 604/270; 604/910; 604/913

(57) ABSTRACT

Delivery devices are described that include (a) a catheter having a first end and a second end; and (b) an expandable, digestible member connected to the first end of the catheter. Methods of delivering liquids and/or nutrition to a patient are also described, which include (a) intubating the patient with a delivery device containing a catheter having a first end and a second end; and an expandable, digestible member connected to the first end of the catheter; and (b) delivering the liquid through the catheter into the patient.

### FIELD OF THE INVENTION

The present invention relates to delivery devices for delivering liquids to patients and, more particularly, to enteral feeding tubes for delivering nutrition to patients via the nose and/or via abdominal openings.

### BACKGROUND

Enteral feeding refers to methods of providing nutrition through a tube directly into the stomach and/or small intestine of a patient. Such methods are frequently the primary or exclusive source of nutrition for patients who cannot safely ingest for any reason sufficient quantities of food to satisfy their caloric requirements. Candidates for enteral feeding include: premature babies, infants, children, and others having limited or compromised esophageal strength/control; the chronically and/or terminally ill; stroke victims; comatose patients; burn victims; cancer patients; those inflicted with Alzheimer's disease; post-operative patients; patients requiring mechanical ventilation; the weak or infirm; and the like.

Typically, enteral feeding devices are inserted into patients through the nose or through an opening in the abdominal wall (e.g., gastrostomies) into the stomach. From this point, feeding may be initiated either pre-pylorically into the stomach (i.e., when the end of the feeding device does not extend past the pyloric valve) or post-pylorically (i.e., when the end of the feeding device extends past the pyloric valve into a region of the small intestine).

Pre-pyloric (intragastric) enteral feeding has the requirement for gastric emptying to deliver nutrients into the small intestine where digestion and absorption takes place. Failed or delayed gastric emptying results in retention of nutrient solution in the stomach, from which it can be regurgitated back into the esophagus. Many of the same patients who require tube feeding due to their inability to eat also have poor gastric function and, therefore, are at risk of esophageal regurgitation and the associated risk of tracheobronchial aspiration. This risk may be reduced using post-pyloric (small bowel) placement of the enteral feeding tube. However, to effect post-pyloric intubation, it is necessary to maneuver the tip of the enteral feeding tube through the pylorus and into the duodenum. Two general approaches have been taken to accomplish this. The one most commonly employed at present is to visually guide the tube, which is often stiffened with an internal wire, through the pylorus using endoscopy or fluoroscopy x-ray techniques. Such methods are inconvenient, costly, and potentially dangerous. Another approach has been to rely on the gastric motility to pass the tube into the small intestine. Tubes currently in use that purport to facilitate passage from the stomach rely on applying weighted tips to flexible tubes. The weights used vary according to design and have included mercury, lead, and tungsten. These tubes have not proven to be effective devices for directing feeding tubes into the small intestine because weight is not functionally important in intestinal passage with the exception of in the esophagus when eating in an upright position. For these reasons, it would be advantageous to develop methods and feeding devices that bypass the use of weighted metal tips and which rely instead on the natural peristaltic motion of the stomach to safely transport the end of a feeding tube through the pylorus and into the small intestine.

### SUMMARY

The scope of the present invention is defined solely by the appended claims, and is not affected to any degree by the statements within this summary.

By way of introduction, a first delivery device embodying features of the present invention includes (a) a catheter having a first end and a second end; and (b) an expandable, digestible member connected to the first end of the catheter.

A second delivery device embodying features of the present invention includes (a) a substantially flexible catheter having a first end and a second end, wherein the catheter contains a biocompatible polymer; and (b) an expandable, digestible member connected to the first end of the catheter, which is substantially dehydrated in a collapsed state, and which swells upon contact with gastric juices.

A method of delivering a liquid to a patient includes (a) intubating the patient with a delivery device containing a catheter having a first end and a second end; and an expandable, digestible member connected to the first end of the catheter; and (b) delivering the liquid through the catheter into the patient.

A method of delivering nutrition to a patient includes (a) intubating the patient through a nasal opening with a delivery device containing a substantially flexible catheter having a first end and a second end, wherein the catheter contains a biocompatible polymer; and an expandable, digestible member connected to the first end of the catheter, which is substantially dehydrated in a collapsed state, and which swells upon contact with gastric juices, wherein intubating continues until the first end of the catheter reaches the interior of the stomach of the patient; (b) contacting the expandable, digestible member with gastric juices in the stomach, such that the expandable, digestible member is converted from the collapsed state to a swelled state; (c) moving at least a portion of the first end of the catheter through the pylorus of the patient by peristaltic action of the stomach upon the expandable, digestible member; and (d) delivering the nutrition through the catheter into a region of small intestine of the patient.

### BRIEF DESCRIPTION OF THE DRAWINGS

FIG. 1 shows a perspective view of a first delivery device embodying features of the present invention, which depicts a first expandable, digestible member in accordance with the present invention.

FIG. 2 shows a cross-sectional view of a second expandable, digestible member in accordance with the present invention.

FIG. 3 shows a cross-sectional view of a third expandable, digestible member in accordance with the present invention.

FIG. 4 shows a detailed view of a fourth expandable, digestible member in accordance with the present invention.

FIG. 5 shows a perspective view of a fifth expandable, digestible member in accordance with the present invention.

FIG. 5A shows a perspective view of a sixth expandable, digestible member in accordance with the present invention prior to insertion in a patient.

FIG. 5B shows the expandable, digestible member of FIG. 5A in an expanded, hydrated state.

FIG. 6 shows a partial cross-sectional view of a sixth expandable, digestible member in accordance with the present invention.

FIG. 7 shows a perspective view of a second delivery device embodying features of the present invention.

FIG. 8 shows a perspective view of a third delivery device embodying features of the present invention.

FIG. 9 shows an illustration of a nasoenteric feeding tube embodying features of the present invention intubated in a patient prior to passage of the tube through the pylorus and prior to degeneration of the erodable coating surrounding the expandable, digestible member.

FIG. 10 shows an illustration of the nasoenteric feeding tube of FIG. 9 after degeneration of the erodable coating and subsequent swelling of the expandable, digestible member but prior to passage of the tube through the pylorus.

FIG. 11 shows an illustration of the nasoenteric feeding tube of FIGS. 9 and 10 after peristaltic transportation of the expandable, digestion member through the pylorus and prior to full digestion of the expandable, digestible member.

### DETAILED DESCRIPTION

Delivery devices and methods suitable for delivering liquids and/or nutrients directly into the small intestine of a patient, either through a nasal opening or an abdominal opening, have been discovered which circumvent the above-mentioned problems. The devices contain (a) a catheter having a first end and a second end, and (b) an expandable, digestible member annealed to the first end of the catheter. The expandable, digestible member, which is in a collapsed state when the patient is initially intubated, functions in a manner analogous to conventional tungsten or silicon weighted tips by helping to guide the tube to its destination, albeit by relying on tractable hydrated mass or volume. It is well established that a small length of yarn connected to a length of string will quickly pass from the stomach into the small intestine, which is the basis of the “sting test” method for collecting small intestinal juices for diagnostic analysis. The above-described tube design relies on a similar principle. By annealing a swelling biopolymer to the first end of a small caliber and highly flexible feeding tube, yarn on the end of a length of string may be emulated. Upon contacting the gastric juices of the stomach, the expandable, digestible member swells. Once swollen, the expandable, digestible member may be engaged and transported by the natural peristaltic motion of the stomach through the pylorus and into the small intestine. However, it would be disadvantageous for the tractable member to remain in place after the tip of the tube has reached its desired position because the intestinal motility would continue to tug on it, which would be uncomfortable to the patient and could result in dangerous consequences. Therefore, designs in accordance with the present invention include the use of a digestible biopolymer as the swelling member. The expandable, digestible member is digested (e.g., in the small intestine) and gradually decreases in size. When the expandable, digestible member is completely digested or at least sufficiently reduced in size, peristaltic transportation of the catheter through the body ceases and enteral feeding through the catheter can be initiated.

Throughout this description and in the appended claims, the following definitions are to be understood:

The phrases “connected to” and “annealed to” refer to all manner of contact between two adjacent elements. The connection may be a physical or chemical bond (e.g., such as would be provided by sulfhydryl bond formation, adhesives, etc.) between two elements made of different materials (e.g., an expandable, digestible member and a first end of the catheter). Alternatively, the connection may be a physical or chemical bond between two structurally contiguous elements formed of the same material (e.g., the first end and remaining portions of the catheter and a flexible linker intermediate therebetween, such as are described below).

The phrase “substantially dehydrated” refers to any state in which the moisture that would normally be present in a material under ambient conditions is no more than about 10% of the moisture the substance would acquire if submerged for a period in water.

The term “fibrous” refers to a thread-like texture.

The phrase “collapsed state” refers to the state of an expandable, digestible member that is substantially dehydrated.

The term “swelled” and the phrase “swelled state” refer to the state of an expandable, digestible member that has increased in size relative to a collapsed state through contact with the contents (e.g., gastric juices) of a stomach.

The term “liquid” refers to any fluid substance including but not limited to pure substances (e.g., water), homogeneous solutions containing one or a plurality of solutes dissolved therein, heterogeneous suspensions, emulsions or multi-phase mixtures, and the like.

The term “intubating” refers to insertion of any portion of a delivery device embodying features of the present invention into any suitable orifice of a patient. Suitable orifices include but are not limited to the nose and abdominal openings.

The phrase “proximal small intestine” refers to any region in the first one third of the length of the small intestine and includes the duodenum, which is the first region of the small intestine located between the stomach and the jejunum.

A first series of presently preferred delivery devices embodying features of the present invention is shown in FIGS. 1-6. The delivery device 2 includes (a) a catheter 4 having a first end 6 and a second end 8; and (b) an expandable, digestible member 10 connected to the first end 6 of the catheter 4.

The catheter 4 may be flexible or rigid and may be manufactured from any biocompatible material including but not limited to polymers, metals, and a combination thereof. It is presently preferred that the catheter be flexible and that that the catheter be comprised of a polymer, more preferably, a biocompatible thermoplastic polymer. Representative biocompatible polymers for use in accordance with the present invention include but are not limited to polytetrafluoroethylene, polyurethane, silicone, and the like, and combinations thereof. While the type of material, dimensions (e.g., length, internal and external diameters, etc.) and degree of flexibility of a catheter used in accordance with the present invention are not limited, it is presently preferred that the catheter be sufficiently slender, flexible, and resilient to be useful for nasoenteric applications.

It is to be understood that catheters in accordance with the present invention may vary considerably with respect to their design. For example, depending on a given application (e.g., whether the tube is a nasoenteral tube or a jejunostomie tube), catheters may vary in length; thickness; interior bore size; external markings (e.g., tubes may be marked with reference distances to assist in intubating a patient); number, shape (e.g., circular, elliptical, etc.), and location of openings in the catheter through which materials may pass from an interior to an exterior thereof or vice versa; type of connector attached at the end of the catheter opposite to the end inserted in the patient; and so forth.

The expandable, digestible member 10 is substantially dehydrated when in a collapsed state but swells upon contact with gastric juices. The duration of contact with gastric juices required to achieve full swelling of the expandable, digestible member 10 may vary according to the nature of the expandable, digestible member 10, the type of weave and of texture thereof, the content and/or acidity of an individual's stomach, and the like. Preferably, full swelling occurs within about 5 minutes of introducing the exposed expandable, digestible member 10 into a stomach, and more preferably within about 3 minutes. In addition, it is preferred that the expandable, digestible member be substantially fibrous. Suitable materials for use as expandable, digestible members in accordance with the present invention include but are not limited to protein collagens (e.g., extracted from cowhide) and carbohydrate polymers (e.g. plant fiber).

Preferably, an external diameter 20 of the expandable, digestible member 10 in a collapsed state does not exceed an external diameter 22 of the catheter 4 by more than about twenty percent, more preferably by more than about ten percent. Furthermore, an external diameter 24 of the expandable, digestible member 10 in a swelled state does exceed an external diameter 22 of the catheter 4 by more than about fifty percent, more preferably by more than about two hundred percent.

It is to be understood that expandable, digestible members embodying features of the present invention may vary considerably with respect to their size and shape both in the collapsed state and in the swelled state. Strictly by way of illustration, the expandable, digestible member may have any regular or irregular geometric shape including but not limited to: spherical, hemispherical, obround, tetrahedral, cubic, cuboidic, pyramidal, frusto-pyramidal, cylindrical, frustoconical, ellipsoidal, oblate spheroidal, prolate spheroidal, catenoidal, and the like. Likewise, it is to be understood that the general shape of the expandable, digestible member may or may not be retained during the transformation from collapsed state to swelled state. Indeed, it is expected that the material will string out from the first end of the feeding tube in a manner similar to yarn.

All manner of configurations and all manner of connection means have been contemplated for joining the expandable, digestible member 10 to the first end 6 of the catheter 4. FIGS. 1-5 depict alternative types of configurations that may be used in accordance with the present invention.

FIG. 1 shows a first embodiment of an expandable, digestible member 10 embodying features of the present invention, whereby the expandable, digestible member 10 contacts only an external edge surface 12 of the first end 6 of catheter 4.

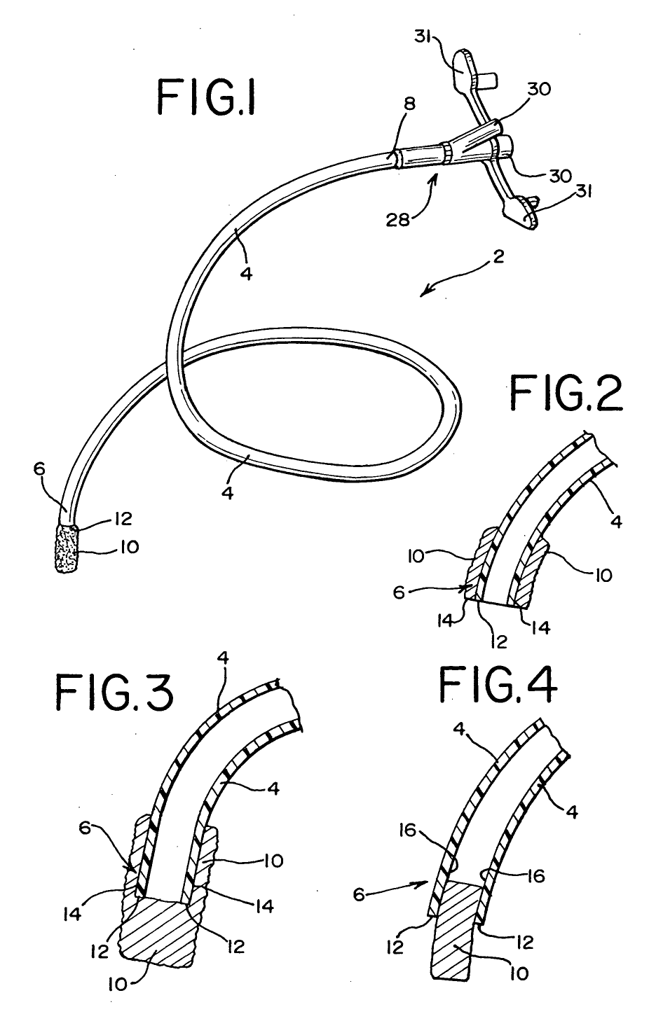

FIG. 2 shows a second embodiment of an expandable, digestible member 10 embodying features of the present invention, whereby the expandable, digestible member 10 contacts only an external side surface 14 of the first end 6 of catheter 4.

FIG. 3 shows a third embodiment of an expandable, digestible member 10 embodying features of the present invention, whereby the expandable, digestible member 10 contacts an external edge surface 12 and an external side surface 14 of the first end 6 of catheter 4.

FIG. 4 shows a fourth embodiment of an expandable, digestible member 10 embodying features of the present invention, whereby the expandable, digestible member 10 contacts an internal side surface 16 of the first end 6 of catheter 4.

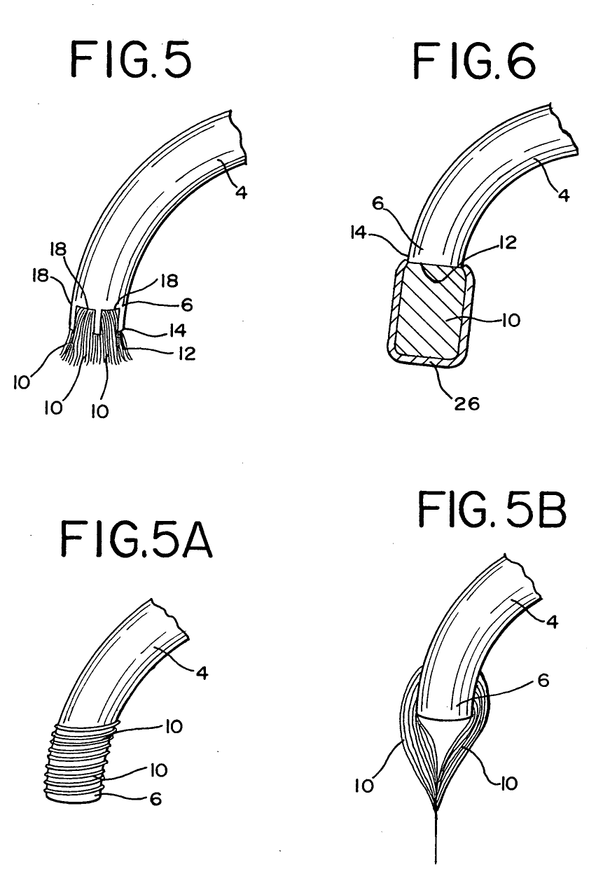

FIG. 5 shows a fifth embodiment of an expandable, digestible member 10 embodying features of the present invention, whereby an external side surface 14 of the first end 6 of catheter 4 defines a plurality of perforations 18, wherein the expandable, digestible member 10 is secured to the first end 6 by being woven through and/or tied to perforations 18.

FIG. 5A shows a sixth embodiment of an expandable, digestible member 10 embodying features of the present invention in which a plurality of strands of the material are anchored to and wrapped around the sides of catheter 4 (e.g., via sulfhydryl bond formation) prior to insertion in a patient. FIG. 5B shows the expandable, digestible member 10 of FIG. 5A in an expanded, hydrated state, wherein the strands of the material have become unwrapped and extend downward into the patient to form a substantially flame-shaped mass. In a first presently preferred embodiment, the length of the strands is between about 3 and about 4 centimeters, although it is to be understood that additional lengths may be used and the ratio of the length of the strands to the tube size may be optimized for best traction.

A presently preferred technique for attaching the expandable, digestible member 10 to the catheter 4 is through the use of a biocompatible adhesive, such as is know to those of ordinary skill in this art. Representative biocompatible adhesives that may be used in accordance with the present invention include but are not limited to fluorinated ethylene propylene.

Other arrangements whereby the expandable, digestible member 10 can be secured to the first end 6 of catheter 4 can be used in accordance with the claimed invention. For example, the expandable, digestible member 10 can be taped to the external side surface 14 with a biocompatible adhesive tape. Alternatively, the external side surface 14 may contain one or more crimped surfaces whereby a portion of an expandable, digestible member 10 is retained. Alternatively, when the catheter is comprised of a metal, an electrodeposition technique for deposition of collagen materials (e.g., such as is described in U.S. Pat. No. 6,391,052) may be employed.

Delivery devices embodying features of the present invention preferably contain an optional erodable coating 26, as shown in FIG. 6, that surrounds at least a portion of, more preferably the entirety of, the expandable, digestible member 10. The erodable coating 26 degenerates upon contacting contents of a stomach and functions to prevent premature swelling of the expandable, digestible member 10 during intubation of a patient. Premature swelling refers to swelling that occurs prior to the arrival of first end 6 in the interior region of a patient's stomach. Suitable materials for use as erodable coatings in accordance with the present invention include water soluble coatings (e.g., sugar-based materials), gelatin, and the like. Optionally, the erodable coating 26 may contain a topical analgesic such that irritation caused by contact between an interior surface of a patient and a portion of the delivery device being inserted may be alleviated.

The second end 8 of catheter 4 may be fitted with a connector 28 of a type known in the art (e.g., see U.S. Pat. No. 4,781,704: col. 3, line 59 to col. 4, line 17) and shown in FIG. 1, whereby nutrient-containing liquids can be introduced into enteral feeding tubes. The connector 28 may contain one or a plurality of ports 30, at least one of which is preferably a female-type adapter. Connectors containing two ports are preferably arranged in a Y-shaped configuration, as shown in FIG. 1. Connectors containing three ports are preferably arranged in a W-shaped configuration (not shown). When ports 30 are not in use, they may be closed off with caps 31.

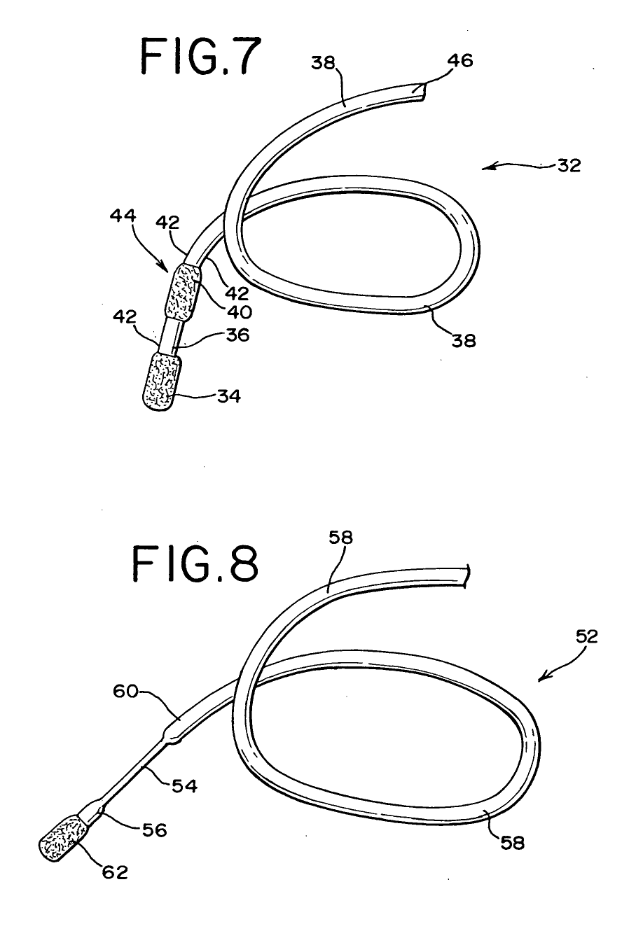

A second series of presently preferred delivery devices embodying features of the present invention is shown in FIG. 7. The delivery device 32 includes a first expandable, digestible member 34 located at the first end 36 of a catheter 38 as well as a second expandable, digestible member 40 connected to an external surface 42 of catheter 38 at a region 44 interposed between the first end 36 and second end 46 thereof. Such devices provide an additional control mechanism whereby a feeding tube may be intubated to a precise location within a patient. For example, the first expandable, digestible member 34 may be coated with a first erodable coating 48 (not shown) and the second expandable, digestible member 40 may be coated with a second erodable coating 50 (not shown) different from the first erodable coating 48. The expandable, digestible member coated with the more rapidly degraded erodable coating will be the first to be exposed to the stomach contents and, therefore, the first to swell and be carried by peristaltic movement through the pylorus. Subsequent degradation of the remaining erodable coating will provide a second handle for further peristaltic transportation of the feeding tube deeper into the duodenum of the patient if so desired.

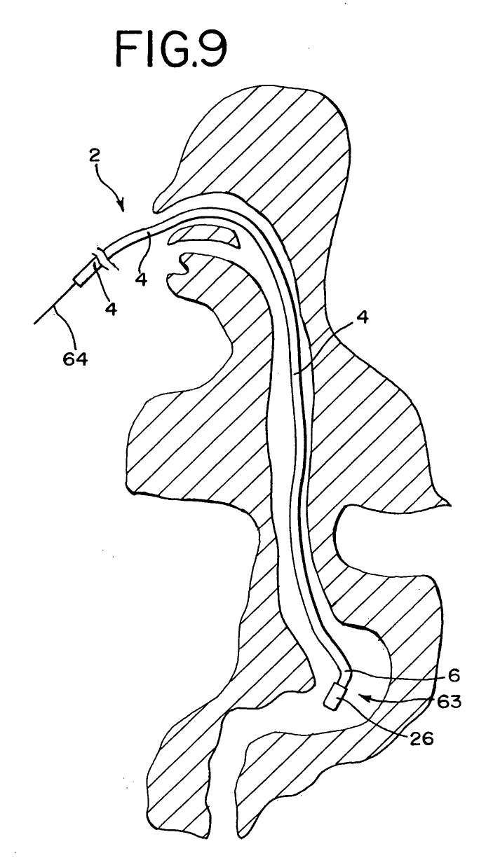

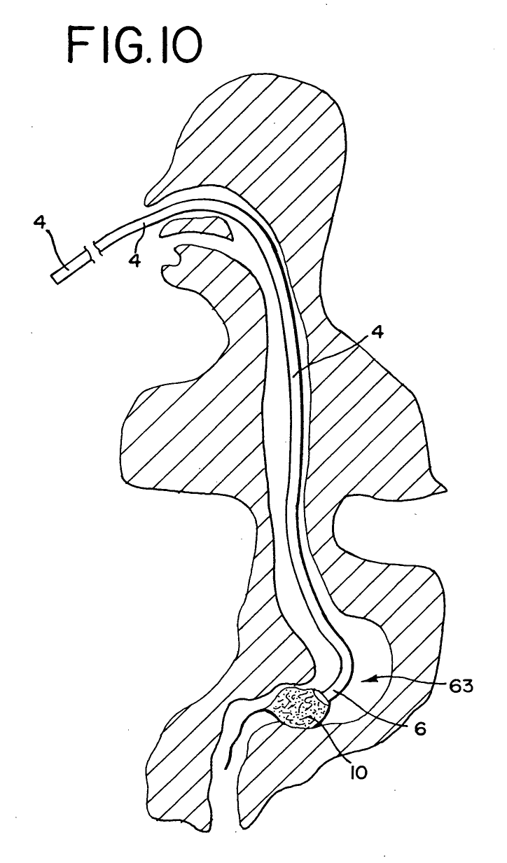

A third series of presently preferred delivery devices embodying features of the present invention is shown in FIG. 8. The delivery device 52 contains a flexible linker 54, wherein the first end 56 of the catheter 58 is connected to remaining portions 60 thereof through the intermediacy of the flexible linker 54. The flexible linker 54 preferably exhibits high deformability and flexibility, such that peristaltic movement of the stomach will respond to the first end 56 which contains an erodable, digestible member 62 as if it were a free-floating, independent mass. The principles by which the flexible linker 54 contributes to post-pyloric intubation are set forth in U.S. Pat. No. 5,057,091 to Erik Anderson, the entire contents of which are incorporated herein by reference, except that in the event of any inconsistent disclosure or definition from the present application, the disclosure or definition herein shall be deemed to prevail.

A first series of presently preferred methods for delivering liquids to a patient include (a) intubating the patient with a delivery device of a type described above, and (b) delivering the liquid through the catheter into the patient. Intubating may be achieved through any suitable orifice and/or surgical opening in a patient, including but not limited to a nasal cavity and an abdominal opening. Preferably, intubating is achieved through the nose or a nasal passage. In addition, it is presently preferred that the liquid be delivered directly into the proximal small intestine of the patient (i.e., post-pylorically).

FIG. 9 illustrates the first phase of intubating a patient in accordance with the present invention. During this phase, the first end 6 of a catheter 4 is guided to the interior of a patient's stomach 63. It is preferred that the delivery device 2 initially contain a stylet (or guide wire) 64, which is removably inserted in the interior bore of catheter 4, and which can be withdrawn therefrom after the first end 6 has been properly positioned.

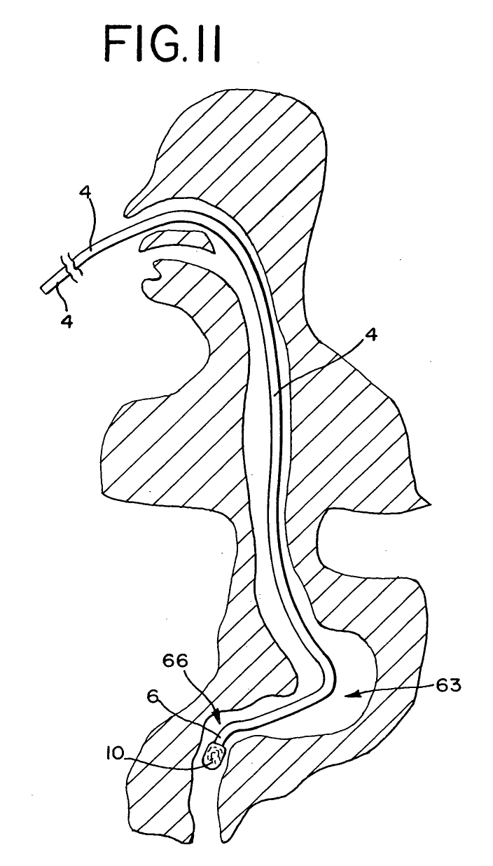

FIG. 10 illustrates the second phase of intubating a patient in accordance with the present invention. During this phase, the erodable coating 26 has been degenerated such that the expandable, digestible member 10 contacts the gastric juices in the stomach and is converted from the collapsed state to the swelled state. As shown in FIG. 10, the hydrated and expanded mass of biomaterial 10 is substantially flame-shaped with a bulky body and a longish tail trailing off into the intestine.

FIG. 11 illustrates the third phase of intubating a patient in accordance with the present invention. During this phase, the swelled expandable, digestible member 10 has been transported by the agency of the peristaltic contractions of the stomach, such that at least a portion of the first end 6 of catheter 4 extends past the pylorus 66 of the patient.

During the final phase of intubation (not shown), expandable, digestible member 10 is gradually digested until an insufficient quantity remains to continue the peristaltic transportation of the catheter 4. In general, traction of the catheter 4 is proportional to the amount of expandable, digestible member 10 that remains undigested.

Although the liquid delivered to patients by delivery devices embodying features of the present invention has been described primarily in reference to enteral feeding and nutrient delivery, it is to be understood that all manner of materials suitable for internal administration to a patient have been contemplated for use in accordance with the present invention. Suitable materials include but are not limited to nutrient solutions, antibiotics, saline solution, analgesics, antiviral agents, antimicrobials, antifungals, anti-tumor drugs, anti-inflammatory agents, antihelmintics, and the like, and combinations thereof.

The foregoing detailed description and accompanying drawings have been provided by way of explanation and illustration, and are not intended to limit the scope of the appended claims. Many variations in the presently preferred embodiments illustrated herein will be obvious to one of ordinary skill in the art, and remain within the scope of the appended claims and their equivalents.

1. A delivery device comprising:
a catheter having a first end and a second end; and
an expandable, digestible member connected to the first end of the catheter.

2. The invention of claim 1 wherein the expandable, digestible member contacts only an external edge surface of the first end of the catheter.

3. The invention of claim 1 wherein the expandable, digestible member contacts only an external side surface of the first end of the catheter.

4. The invention of claim 1 wherein the expandable, digestible member contacts an external edge surface and an external side surface of the first end of the catheter.

5. The invention of claim 1 wherein the expandable, digestible member contacts an internal side surface of the first end of the catheter.

6. The invention of claim 1 the expandable, digestible member is substantially dehydrated when in a collapsed state, and wherein the expandable, digestible member swells upon contact with gastric juices.

7. The invention of claim 1 wherein the expandable, digestible member is substantially fibrous.

8. The invention of claim 1 wherein the expandable, digestible member comprises collagen.

9. The invention of claim 1 wherein the expandable, digestible member comprises meat.

10. The invention of claim 1 wherein an external diameter of the expandable, digestible member in a collapsed state does not exceed an external diameter of the catheter by more than about twenty percent.

11. The invention of claim 1 wherein an external diameter of the expandable, digestible member in a collapsed state does not exceed an external diameter of the catheter by more than about ten percent.

12. The invention of claim 1 wherein an external diameter of the expandable, digestible member in a swelled state does not exceed an external diameter of the catheter by more than fifty percent.

13. The invention of claim 1 wherein an external diameter of the expandable, digestible member in a swelled state does not exceed an external diameter of the catheter by more than thirty percent.

14. The invention of claim 1 wherein the catheter is substantially flexible.

15. The invention of claim 1 wherein the catheter is substantially rigid.

16. The invention of claim 1 wherein the catheter comprises a material selected from the group consisting of polymers, metals, and a combination thereof.

17. The invention of claim 1 wherein the catheter comprises a biocompatible thermoplastic polymer.

18. The invention of claim 1 wherein the catheter comprises a polymer selected from the group consisting polytetrafluoroethylene, polyurethane, silicone, and combinations thereof.

19. The invention of claim 1 further comprising at least one secondary expandable, digestible member connected to an external surface of the catheter intermediate between the first end and the second end.

20. The invention of claim 1 wherein the expandable, digestible member is connected to the first end by a biocompatible adhesive.

21. The invention of claim 1 wherein a side surface of the first end defines a plurality of perforations, and wherein the expandable, digestible member is connected to the first end through at least one of the perforations.

22. The invention of claim 1 wherein a side surface of the first end comprises means for securing the expandable, digestible member.

23. The invention of claim 1 further comprising an erodable coating surrounding at least a portion of the expandable, digestible member, whereby the erodable coating degenerates upon contacting contents of a stomach.

24. The invention of claim 23 wherein the erodable coating comprises a topical analgesic whereby irritation caused by insertion of a portion of the delivery device into a patient is alleviated.

25. The invention of claim 1 further comprising a connector through which the catheter may be loaded, wherein the connector is connected to the second end of the catheter and wherein the connector comprises at least one port.

26. The invention of claim 25 wherein the connector comprises a plurality of ports.

27. The invention of claim 26 wherein the connector is selected from the group consisting of a Y-shaped connector comprising two ports and a W-shaped connector comprising three ports.

28. The invention of claim 26 wherein at least one of the ports comprises a female adapter.

29. The invention of claim 1 further comprising a flexible linker, wherein the first end of the catheter is connected to remaining portions of the catheter through intermediacy of the flexible linker.

30. A delivery device comprising:
a substantially flexible catheter having a first end and a second end, wherein the catheter comprises a biocompatible polymer; and
an expandable, digestible member connected to the first end of the catheter, which is substantially dehydrated in a collapsed state, and which swells upon contact with gastric juices.

31. The invention of claim 30 further comprising an erodable coating surrounding the expandable, digestible member, whereby the erodable coating degenerates upon contacting contents of a stomach.

32. A method of delivering a liquid to a patient comprising:
intubating the patient with a delivery device comprising a catheter having a first end and a second end; and an expandable, digestible member connected to the first end of the catheter; and
delivering the liquid through the catheter into the patient.

33. The invention of claim 32 wherein the intubating is through a nasal opening of the patient.

34. The invention of claim 32 wherein the intubating is through an abdominal opening of the patient.

35. The invention of claim 32 wherein the liquid comprises a nutrient.

36. The invention of claim 32 wherein the intubating is continued until the first end of the catheter reaches an interior of a stomach of the patient.

37. The invention of claim 36 further comprising contacting the expandable, digestible member with gastric juices in the stomach, such that the expandable, digestible member is converted from a collapsed state to a swelled state.

38. The invention of claim 37 further comprising moving at least a portion of the first end of the catheter through the pylorus of the patient by peristaltic action of the stomach on the expandable, digestible member in the swelled state.

39. The invention of claim 38 further comprising digesting at least a portion of the expandable, digestible member.

40. The invention of claim 39 wherein movement of the catheter through the pylorus is proportional to an amount of the expandable, digestible member remaining undigested.

41. The invention of claim 32 wherein the delivery device further comprises an erodable coating surrounding the expandable, digestible member, whereby the erodable coating degenerates upon contacting contents of a stomach of the patient.

42. A method of delivering nutrition to a patient comprising:
intubating the patient through a nasal opening with a delivery device comprising:
a substantially flexible catheter having a first end and a second end, wherein the catheter comprises a biocompatible polymer; and
an expandable, digestible member connected to the first end of the catheter, which is substantially dehydrated in a collapsed state, and which swells upon contact with gastric juices, wherein intubating continues until the first end of the catheter reaches an interior of a stomach of the patient;
contacting the expandable, digestible member with gastric juices in the stomach, such that the expandable, digestible member is converted from the collapsed state to a swelled state;
moving at least a portion of the first end of the catheter through the pylorus of the patient by peristaltic action of the stomach upon the expandable, digestible member; and
delivering the nutrition through the catheter into a small intestine region of the patient.

43. The invention of claim 42 further comprising digesting at least a portion of the expandable, digestible member, such that movement of the catheter through the pylorus is proportional to how much of the expandable, digestible member remains undigested.

44. The invention of claim 42 wherein the delivery device further comprises an erodable coating surrounding the expandable, digestible member, whereby the erodable coating degenerates upon contacting contents of the stomach, and whereby swelling of the expandable, digestible member is substantially prevented until the erodable coating begins to degenerate.

45. The invention of claim 42 wherein the delivery device further comprises a stylet removably inserted in an interior region of the catheter, and wherein the method further comprises:
guiding the first end of the catheter into the interior of the stomach by use of the stylet; and
removing the stylet.
